(19) United States
(12) Patent Application Publication
(10) Pub. No.: US 2024/0156844 A1
(43) Pub. Date: May 16, 2024

(54) ORALLY ADMINISTRABLE FORMULATION

(71) Applicants: Allen Greenspoon, Hamilton (CA); Anthony Adili, Hamilton (CA)

(72) Inventors: Allen Greenspoon, Hamilton (CA); Anthony Adili, Hamilton (CA)

(21) Appl. No.: 18/506,793

(22) Filed: Nov. 10, 2023

Related U.S. Application Data
(60) Provisional application No. 63/383,171, filed on Nov. 10, 2022.

(51) Int. Cl. A61K 31/675; A61K 9/00; A61K 9/06; A61K 31/00; A61K 31/196; A61K 31/485; A61K 31/575; A61K 38/46; A61K 47/10; A61K 47/20; A61K 47/24; A61K 47/36; A61K 47/38; A61K 47/44

(52) U.S. Cl. CPC A61K 31/675; A61K 9/006; A61K 9/06; A61K 31/196; A61K 31/485; A61K 31/575; A61K 31/658; A61K 38/465; A61K 47/10; A61K 47/20; A61K 47/24; A61K 47/36; A61K 47/38; A61K 47/44; C12Y 301/01003

(57) ABSTRACT

An orally administrable formulation is provided. The formulation comprises a hydrophilic polymeric base incorporating a pharmaceutical component. The pharmaceutical component comprises a pharmaceutical compound selected from a cannabinoid, a psychedelic compound or a mixture thereof and an absorption enhancer to enhance absorption of the pharmaceutical agent by the oral mucosa.

### FIELD OF THE INVENTION

The present invention relates to orally administrable formulations useful to deliver pharmaceutical agents.

### BACKGROUND

Many people worldwide are afflicted with psychological or mood disorders, such as depression, anxiety, compulsion, and post-traumatic stress disorders. Many of these conditions are believed to involve a person's serotonin system-including interactions between the neurotransmitter serotonin (often abbreviated 5-HT), and several different subtypes of serotonin neurotransmitter receptors found in the human body.

However, despite their unquestionable popularity and commercial success, the users of these pharmaceutical products are unsatisfied with their long onset times, severe side-effects, and poor efficacy. In many cases, these drugs are harmful to the user. For example, many people taking prescription drugs targeting serotonin report feeling suicidal thoughts, sexual dysfunction, fatigue, elevated blood pressure, blurred vision, abnormal heart rate, nausea, and weight gain.

Psychedelic compounds have recently made their way back into mainstream medical research. Psychedelic compounds such as (5R,8R)-(+)-lysergic acid-N,N-diethylamide (LSD), psilocybin, N,N-dimethyltryptamine (DMT), ibogaine and mescaline have been of great interest based on their apparent high efficacy for treating mental afflictions and their impressive safety profile. “Classic” psychedelics generally refer to LSD, psilocybin, mescaline, ibogaine, DMT, and 5-methoxy-N,N-dimethyltryptamine (5-MeO-DMT) and are typically defined by their indole-containing molecular structure as illustrated in FIG. 1.

Psilocybin (also known as 4-phosphoryloxy-N,N-dimethyltryptamine or [3-(2-trimethylaminoethyl)-1H-indol-4-yl] dihydrogen phosphate) is considered to be the most abundant psychoactive compound within a number of fungal species. Psilocin (4-hydroxy-N,N-dimethyltryptamine) is considered to be the second most abundant compound Formulated and administered correctly, psilocin and psilocybin have been found to provide fast-acting and long-lasting changes to a person's mood. These effects can be accomplished with only minor side effects, low potential for addiction, low potential for abuse, and low risk of toxicity.

Major barriers to developing oral formulations of larger molecules such as psychedelic compounds include poor intrinsic permeability, lumenal and cellular enzymatic degradation, rapid clearance, and chemical instability in the gastrointestinal (GI) tract. Approaches effective to address these barriers in formulations comprising small, organic drug molecules, are not readily applied to formulations including psychedelic compounds.

Oral administration routes for large molecules has received far more attention than other administrable routes. In addition, to the fact that the oral cavity is easily accessible and convenient, oral membranes such as the sublingual mucosa and the buccal mucosa, are relatively permeable, thereby providing ready absorption of orally administered drugs, and thus, acceptable bioavailability. However, as the ability of molecules to permeate through the oral mucosa appears to be related to molecular size, lipid solubility and charge, molecular permeability of molecules greater than 1000 daltons is significantly decreased. While some penetration enhancing products have been determined to facilitate mucosal administration of large molecule drugs, very few such enhancers have been approved for market use due to lack of a satisfactory safety profile, lowering of mucosal barrier function, impairment of the mucociliary clearance protective mechanism, and irritant properties. In addition, penetration enhancers are extremely bitter and unpleasant in taste.

Thus, it would be desirable to develop a formulation effective for the delivery of therapeutic compounds, including for example, psychedelic compounds.

### SUMMARY

A novel orally administrable formulation is provided herein.

In one aspect of the invention, an orally administrable formulation is provided comprising a hydrophilic polymer base incorporating a pharmaceutical component comprising a pharmaceutical agent or a mixture of pharmaceutical agents and an absorption enhancer.

In another aspect of the invention, an orally administrable formulation is provided comprising a hydrophilic polymer base incorporating a pharmaceutical component comprising a pharmaceutical agent, or a mixture of pharmaceutical agents, an enzymatic absorption enhancer and a non-enzymatic absorption enhancer.

These and other aspects of the invention are described herein.

### DETAILED DESCRIPTION

A novel orally administrable formulation is provided herein comprising a hydrophilic polymer base admixed with a pharmaceutical component comprising a pharmaceutical agent combined with an absorption enhancer.

The formulation may include any one of a number of pharmaceutical agents, or mixtures thereof. For example, the pharmaceutical agent may include one or more of:

The composition is not particularly restricted with respect to the pharmaceutical agent. Examples of pharmaceutical agents that may be incorporated in the present formulation include, but are not limited to:
antimicrobial agents, such as triclosan, cetyl pyridium chloride, domiphen bromide, quaternary ammonium salts, zinc compounds, sanguinarine, fluorides, alexidine, chlorhexidine, octonidine, EDTA, and the like;
non-steroidal anti-inflammatory drugs (NSAIDs), such as aspirin, acetaminophen, ibuprofen, ketoprofen, diflunisal, fenoprofen calcium, naproxen, diclofenac, tolmetin sodium, indomethacin, and the like;
anti-tussives, such as benzonatate, caramiphen edisylate, menthol, dextromethorphan hydrobromide, chlophedianol hydrochloride, and the like;
decongestants, such as pseudoephedrine hydrochloride, phenylepherine, phenylpropanolamine, pseudoephedrine sulfate, and the like;
anti-histamines, such as brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, cetirizine, levo cetirizine and the like;
expectorants, such as guaifenesin, ipecac, potassium iodide, terpin;
anti-diarrheals, such a loperamide, and the like;
H2-antagonists, such as famotidine, ranitidine, and the like;
proton pump inhibitors, such as omeprazole and lansoprazole;
nonselective CNS depressants, such as aliphatic alcohols, barbiturates and the like;
nonselective CNS stimulants such as caffeine, nicotine, nicotine polacrilex, nicotine in combination with alkaline agents, strychnine, picrotoxin, pentylenetetrazol and the like;
drugs that selectively modify CNS function such as phenyhydantoin, phenobarbital, primidone, carbamazepine, ethosuximide, methsuximide, phensuximide, trimethadione, diazepam, benzodiazepines, phenacemide, pheneturide, acetazolamide, sulthiame, bromide, and the like;
anti-parkinsonism drugs such as levodopa, amantadine and the like;
analgesic-antipyretics such as salycilates, phenylbutazone, indomethacin, phenacetin and the like;
psychopharmacological drugs such as chlorpromazine, methotrimeprazine, haloperidol, clozapine, reserpine, imipramine, tranylcypromine, phenelzine, MC-4 receptor antagonist, lithium and the like;
hypnotics, sedatives, antiepileptics, awakening agents;
vitamins and minerals;
amino acids and peptides;
sildenafil citrate;
PPY (3-36); deca-peptide; KSL-W (acetate), fluor
antidiabetic drugs, e.g. metformin, metformin HCL, glyburide and insulin secretart agent, insulin stimulators, fat metabolizers, carbohydrates metabolizers, insulin, cholesterol lowering agents like statins, exenatide, GLP-1, etc.
opioid analgesics such as alfentanil, allylprodine, alphaprodine, anileridine, benzylmorphine, bezitramide, buprenorphine, butorphanol, clonitazene, codeine, cocaine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazine, fentanyl, heroin, hydrocodone, hydromorphone, oxymorphone, hydromorphine, naltrexone, naloxone, 14-hydroxycodeinone, neopinone, hydroxypethidine, isomethadone, ketobemidone, levallorphan, levorphanol, levophenacylmorphan, lofentanil, meperidine, meptazinol, metazocine, methadone, metopon, morphine, diamorphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tramadol, tilidine, mixed mu-agonists/antagonists, mu-antagonist combinations, mixtures of any of the foregoing, and the like. The opioid analgesic may be in the form of the free base, or in the form of a pharmaceutically acceptable salt, or in the form of a pharmaceutically acceptable complex;
terpene and/or terpenoid from plant or fungal sources, including but not limited to, myrcene, beta caryophyllene, pinene, limonene, terpinolene, humulene, nerolidol, linalool, ocimene, guaiol, bisabolol, alpha phellandrene, cadinene, camphene, camphor, citral, citronellol, delta 3-carene, eucalyptol, eugenol, gamma terpinene, geraniol, humulene, nerol, nerolidol, ocimene, para-cymene, phytol, pulegone, terpineol, valencene, mixtures thereof or pharmaceutically acceptable salts thereof,
a “cannabinoid” or “cannabinoid derivative” which refers to herein a class of diverse chemical compounds that act on cannabinoid receptors, e.g. cannabinoid receptor type 1 (CB1) and cannabinoid receptor type 2 (CB2), in cells that repress neurotransmitter release in the brain. Cannabinoids include the endocannabinoids (produced naturally in the body by humans and animals, such as arachidonoyl-ethanolamide (anandamide), 2-arachidonoyl glycerol (2-AG) and arachidonyl glyceryl ether (noladin ether)); the phytocannabinoids (found in cannabis and some other plants such as tetrahydrocannabinol (THC), cannabidiol (CBD) and cannabinol (CBN); synthetic cannabinoids (manufactured artificially), and functionally equivalent derivatives and analogues of any of these. Examples of cannabinoids include, but are not limited to, cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), naphthoylindoles such as JWH-018, JWH-073, JWH-398, JWH-200, JWH-081, 4-methyl-JWH-073, JWH-015, JWH-122, JWH-220, JWH-019, JWH-007; phenylacetylindoles such as JWH-250 and JWH-203; benzoylindoles such as RCS-4, AM-694 and WIN 48,098; cyclohexylphenoles such as CP 47,497-C8 and CP 47,497; HU-210 and 3-dimethylnepty 11 carboxylic acid homologine 8. Cannabinoids also include tetrahydrocannabinoids and analogs thereof, namely, delta-9 tetrahydrocannabinol (THC or dronabinol) and functionally equivalent compounds, including analogs and derivatives thereof such as delta-8 tetrahydrocannabinol (D8-THC), tetrahydrocannabinol acid (THCA), tetrahydrocannabivarin (THCV), tetrahydrocannabivarin acid (THCVA), nabilone, rimonabant (SR141716), JWH-018, JWH-073, CP-55940, dimethylheptylpyran, HU-210, HU-331, SR144528, WIN 55, 212-2, JWH-133, levonantradol, and AM-2201. Mixtures of any of the above cannabinoids is also encompassed. The term “functionally equivalent” as it relates to analogs and derivatives of a cannabinoid refers to compounds which bind a cannabinoid receptor, and/or which exhibit the same or similar therapeutic effect, e.g. at least about 50% of the activity of the cannabinoid from which it is derived;
fungal polysaccharides, polysaccharide-protein or -peptide complexes (PSP's), glycoproteins, proteoglycans, alpha and beta glucans, heteroglycans, peptidoglycans, or lectins, having therapeutic activity such as anti-tumour, immunomodulatory, antioxidant, anti-inflammatory, anti-microbial or anti-diabetic activity;
biological response modifiers (BRM's) such as monoclonal antibodies, interferons, interleukins, tumour necrosis factor, colony stimulating factors and vaccines;
psychedelic compounds, including, for example, indole-containing psychedelic compounds. Examples include, but are not limited to, tryptamines, phenethylamines, ketamine, ibotenic acid, muscimol, salvinorin A or lysergamides, such as DMT (N,N-dimethyltryptamine) which may be in the form of ayahuasca, 5-hydroxy-N,N-dimethyltryptamine (5-HO-DMT), 5-methoxy-N,N-dimethyltryptamine (5-MeO-DMT), LSD (lysergic acid diethylamide), mescaline (3,4,5-trimethoxyphenethylamine), ibogaine, PCP (phenylcyclohexyl piperidine), 4-phosphoryloxy-N,N-dimethyltryptamine (psilocybin) and analogues thereof such as [3-[2-(methylamino)ethyl]-1H-indol-4-yl]dihydrogen phosphate (baeocystin), 4-hydroxy-N,N-dimethyltryptamine (psilocin), amphetamine, 3,4-methylenedioxy amphetamine (MDA), methylenedioxyethylamphetamine (MDEA), MDMA (3,4-methylenedioxy-methamphetamine) and analogues or combinations thereof, including extracts and or compounds extracted from fungal sources such as but not limited to the genera Copelandia, Gymnopilus, Inocybe, Mycena, Panaeolus, Pholiotina, Pluteus or Psilocybe, and other sources such as plant, e.g. cacti, for example, extracts from the Echinopsis and Lophophora genus of cactus containing phenethylamine alkaloids such as mescaline.

The selected pharmaceutical agent(s) may be readily commercially available.

Alternatively, certain pharmaceutical agents may be chemically or biosynthetically synthesized using methods available in the art.

Plant- and fungal-derived compounds such as cannabinoids and terpenes may be obtained using established methods of extraction, or may be chemically synthesized. Cannabinoids and some terpenes may be extracted from a Cannabis plant (e.g. Cannabis sativa, Cannabis indica, and Cannabis ruderali). Terpenes may also be extracted from other plants, such as tea, thyme, sage, and from fruits such as citrus fruits, as well as from fungi. Some of these compounds are also commercially available. In other embodiments, as described in U.S. Pat. No. 9,833,408 (the contents of which are incorporated herein by reference), plant material comprising cannabinoids and/or terpenes may be incorporated directly into the formulation.

Fungal-derived compounds such as psychedelic compounds may be extracted from a fungal product (e.g. a mushroom), and provided as a dried powder or provided as a liquid extract in solvent. Psilocybin may be extracted from mushroom material using alcohol, and subsequently crystallized. Another extraction protocol that may be used is as follows. Pulverized mushroom is mixed with dilute acetic acid in a beaker and titrated to pH 4 with acid (such as concentrated (glacial) acetic acid). Following incubation, this mixture is heated to 70° C. This heating step dephosphorylates psilocybin to psilocin. The acid mixture is separated from the raffinate, brought to pH 8, and extracted, for example using diethyl ether. The extractant is dried to yield psilocin precipitate that may then be crystallized to a white powder, e.g. using a 1:3 ratio of chloroform to heptane.

In other embodiments, the fungal product may be dried and pulverized using established protocols and directly incorporated into the formulation.

Psychedelic compounds may also be chemically or biochemically synthesized for use in the formulation using known chemical synthesis methods.

The pharmaceutical component may comprise two or more pharmaceutical agents. In one embodiment, the two or more pharmaceutical agents may target a particular condition. Thus, each pharmaceutical agent may treat the condition using the same or different modes of action (multi-modal) to thereby enhance the treatment. For example, the present formulation may be designed to treat pain using a multi-modal approach, and thus, may comprise a pharmaceutical component comprising an opioid, an NSAID, acetaminophen, and/or an SSRI antidepressant. Alternatively, the formulation may comprise an analgesic compound, such as an opioid analgesic, in combination with a second pharmaceutical agent selected from a cannabinoid, a terpene or terpenoid and/or a psychedelic drug, or may comprise two or more psychedelic drugs, optionally in combination with a cannabinoid and/or a terpene. Preferred combinations of pharmaceutical agents are combinations which exhibit a synergistic effect, i.e. which exhibit a therapeutic effect which is greater than the additive therapeutic effect of each pharmaceutical agent on its own. In this regard, reduced dosages of at least one of the pharmaceutical agents in the combination results, such as a reduction of the dosage by at least about 10%, and preferably, at least about 20%, 30%, 40%, 50%, 75% or more, in comparison to the dosage of the at least one pharmaceutical agent when used alone. Such a reduced dosage may additionally result in a more favourable or reduced side effect profile. In one embodiment, the dosage of an opioid analgesic is reduced as a result of such a multi-modal combination.

In another embodiment, pharmaceutical agents may be combined within the present formulation which treat different aspects of a particular condition, for example, pain and inflammation, and may additionally incorporate an anti-tumour agent or other pharmaceutical agent. The combination of multiple pharmaceutical agents to treat different aspects of a particular condition may be selected to exhibit a therapeutic effect that is greater than each pharmaceutical agent on its own, as set out above. Examples of pharmaceutical agents that may be combined include an anti-inflammatory agent such as diclofenac, acetaminophen, ibuprofen, ketoprofen or naproxen (or any other anti-inflammatory agent as listed herein), with an opioid analgesic such as morphine, codeine, hydromorphone, oxycodone or fentanyl (or any other opioid analgesic such as those listed herein) and a psychedelic drug such as psilocybin (or any other psychedelic drug as set out herein). The combination may additionally include a cannabinoid or terpene as listed above. An exemplary combination is diclofenac, psilocybin, and an opioid such as morphine or hydromorphone. The combination may be further enhanced with a cannabinoid such as CBD.

The amount of pharmaceutical agent or agents in the present formulation, including combinations thereof, will vary with the particular selected pharmaceutical agent(s), the form in which the pharmaceutical is incorporated within the formulation, the condition to be treated and the effective dosage of each. Generally, the formulation comprises about 0.1-40% by wt of pharmaceutical agent, preferably, 1-30% by wt of pharmaceutical agent. As one of skill in the art will appreciate, the appropriate dosage of pharmaceutical agent will vary with each pharmaceutical agent. In one embodiment, the formulation comprises a cannabinoid in an amount which yields about 1-60 mg of cannabinoid per dosage form. In another embodiment, the formulation comprises a psychedelic drug in an amount which yields about 1-60 mg of psychedelic drug per dosage form.

In further embodiments, the formulation comprises a mixture of pharmaceutical agents in amounts that are therapeutically effective, and which preferably complement or enhance the therapeutic effect of one or more of the pharmaceutical agents. In this regard, the present formulation may incorporate a combination of pharmaceutical agents in an amount of about 0.1 to 100 grams, about 1 to 60-80 grams, or about 5 to 50 grams. The term “about” is used herein to refer to an amount that varies from a listed amount by 10% more or less from the listed amount. As one of skill in the art will appreciate, the amount of each pharmaceutical agent will vary based on the therapeutically effective amount of each. An exemplary multi-modal combination is 10-50 mg of diclofenac, 2-50 mg psilocybin, and 10-30 mg of an opioid such as morphine, or 1-10 mg of the opioid hydromorphone, and optionally, 10-50 mg of CBD. Another exemplary combination is 25 mg diclofenac, 10 mg psilocybin, 20 mg CBD, and 2 mg hydromorphone

The formulation comprises a hydrophilic polymer base. The components of the polymer base may vary substantially depending on the desired masticatory and other sensory characteristics of the final product. Thus, “hydrophilic polymer base” refers to a base of the present formulation comprising hydrophilic elastomeric polymer which is suitable for oral administration, which is dissolvable within the oral cavity (i.e. in aqueous solution), and which is optionally chewable, in combination with one or more components which supplement the properties of the base. Thus, the polymer base releases pharmaceutical agent on dissolution within the oral cavity to be absorbed by the oral mucosa. The polymer base comprises one or a combination of hydrophilic elastomeric polymers, each in an amount of about 1 to 40% by weight of the formulation, preferably, with a total hydrophilic polymeric content of 1-80% by wt, such as 10-60% by wt. of the formulation.

The hydrophilic polymer base comprises one or more hydrophilic elastomeric polymers suitable to form a gel in an aqueous solution. As one of skill in the art will appreciate, the selected hydrophilic polymer(s) will vary with the pharmaceutical agent(s) to be delivered and desired release profile of the pharmaceutical agent(s), e.g. immediate release versus sustained, delayed, pulsed, continuous, controlled, targeted or programmed release. Thus, for immediate release, a rapidly dissolving hydrophilic polymer or combination of polymers is selected for use in the formulation, whereas to achieve sustained or delayed release, a hydrophilic polymer or combination of polymers is selected which dissolves more slowly. To achieve a combination of immediate and delayed release of a combination of pharmaceutical agents, a formulation comprising layers may be utilized. For example, a pharmaceutical agent to be released quickly is formulated in a layer comprising a rapidly dissolving hydrophilic polymer or combination thereof, while a pharmaceutical agent for delayed release is formulated in a layer comprising a hydrophilic polymer or combination thereof which dissolve more slowly.

Examples of suitable gel-forming elastomeric polymers include, but are not limited to, polyethylene glycol, polyacrylamides, polyacrylic acid, acrylic acid, polyacrylic acid copolymer, polyvinyl alcohol, hydroxypropylmethyl cellulose, hydroxyethyl cellulose, hydroxypropyl cellulose, polyvinyl pyrrolidone, carboxymethyl cellulose, glycolide, polylactide, methylmethacrylate copolymer, carboxyvinyl polymer, polysaccharides such as pullulan, amylose, high amylose starch, hydroxypropylated high amylose starch, alginate, pectin, dextrin, dextran, chitin, chitosan, levan, elsinan, scleroglucan and alternant, and combinations thereof in amounts that dissolve, disperse and swell in water to form a weakly cohesive internal structure, e.g. a three dimensional network that spans the volume of the water it is in, to result in a dissolvable film or gel on oral administration.

Additional secondary film forming agents that may be added to the polymer base to optimize tensile strength, stability, flexibility, solubility characteristics and brittleness include agents such xanthan gum, tragacanth gum, guar gum, acacia gum, arabic gum, carrageenan gum, collagen, gelatin, zein, gluten, soy protein isolate, whey protein isolate, casein and mixtures thereof. The amount of secondary film forming agent will vary depending on the amount and nature of the hydrophilic elastomeric polymer, and will generally be in an amount of 0.1 to 10% by wt of the polymer base (excluding water).

A preferred hydrophilic elastomeric polymer is pullulan, in amounts ranging from about 1-80 wt % of the polymer base, preferably from about 25 to about 60 wt %, more preferably from about 30 to about 50 wt % of the polymer base. Another preferred film forming agent is a mixture of pullulan and alginate or hydroxypropylmethyl cellulose, or a mixture of each of these. Preferred secondary film-forming agents include xanthan gum, tragacanth gum, guar gum, acacia gum, arabic gum, gelatin and/or carrageenan gum.

Plasticizers that vary the firmness of the formulation may optionally be added to the polymer base. Examples of plasticizers that may be included in the base include, but are not limited to, n-butyl stearate; oleic acid; mono-, di-, or tri-glyceryl esters of the saturated or unsaturated fatty acids of oleic acid, caprylic acid, butyric acid, capric acid, caproic acid and lauric acid; mineral oil, liquid petroleum hydrocarbons, squalane, squalene, castor oil and other ricinoleate derivatives; diethylene or propylene glycols and derivatives; tributyl acetyl citrate, tributyl citrate, lecithin, coconut oil, glyceryl tributyrate, Zn laurate, calcium stearate, propylene glycol monostearate, propylene glycol monolaurate, fatty acids, glycerine, butyl sebacate, butyl benzyl sebacate, diacetyl tartaric acid esters of mono- and diglycerides of edible fat oils or edible fat forming acids; acetylated monoglyceride; petrolatum, stearyl monoglyceride citrate, limonene, polylimonene, butyl lactate, butyl oleate and mixtures thereof. Plasticizer may be included in the formulation in an amount ranging from about 0-60 wt % of the polymer base, for example, 1-50 wt % or 10-40 wt % of the polymer base

Natural or synthetic resins may optionally be included in the polymer base. Examples include natural rosin esters (often referred to as ester gums), such as glycerol esters of partially hydrogenated rosins, glycerol esters of polymerised rosins, glycerol esters of partially dimerized rosins, glycerol esters of tally oil rosins, penta erythritol esters of partially hydrogenated rosins, methyl esters of rosins, partially hydrogenated methyl esters of rosins, and penta erythritol esters of rosins; and synthetic resins such as terpene resins derived from alpha-pinene, beta-pinene, and/or d-limonene, and natural terpene resin. Resins, including combinations of resins, may be included in the formulation in an amount ranging from 5-10% by wt of the polymer base.

The present formulation may include a softener in addition to a plasticizer in the polymer base Examples of suitable softeners include, but are not limited to, glycerin, edible oil, mannitol and sorbitol. The wax may be included in an amount in the range of about 1-10% by weight of the polymer base.

The polymer base of the present formulation may also, if desired, include one or more fillers/texturizers including as examples, magnesium or calcium carbonate, sodium sulphate, ground limestone, silicate compounds such as magnesium and aluminum silicate, kaolin and clay, aluminum oxide, aluminum phosphate silicium oxide, talc, titanium oxide, mono-, di- and tri-calcium phosphates, cellulose polymers, such as wood fibers, mineral oil such as paraffin oil, plant saponins from Quillaia, soybean or polygala senega, and combinations thereof. The filler is preferably hydrophobic. The amount of filler/texturizer included in the polymer base will be an amount sufficient to yield a polymer base having the desired consistency.

The polymer base of the present formulation may additionally include a water soluble wax. Suitable waxes include microcystalline waxes which contain isoparaffinic (branched) hydrocarbons and naphthenic hydrocarbons such as Microwax™ 1750, Microwax™ 820, Paramelt® HMP (a blend of refined mineral hydrocarbon waxes having a high melting point and a low oil content), Paramelt® LMP (a low melting point, low oil content microwax), and Microwax™ ZG. The wax may be included in an amount in the range of about 1-10% by weight of the polymer base

The polymer base and optional base components of the formulation (e.g. secondary film forming agent(s), plasticizer(s), resin(s), softener(s), filler(s) and waxes), and amounts thereof, will determine the properties of the formulation, and thus may be varied in order to achieve an orally administrable formulation with the desired properties, as will be appreciated by one of skill in the art, for example, solubility characteristics, and drug release profile

In one embodiment, the orally administrable formulation comprises a polymer base comprising one or more hydrophilic elastomeric polymers in an amount in the range of about 45-85% by wt of the base, and preferably, about 50-80% by wt of the base such as about 55% by wt, 60% by wt, 65% by wt, 70% by wt or 75% by wt, along with secondary film-forming agents in an amount of about 5-15% by wt of the base, and a plasticizer in an amount of about 10-40% by wt of the base, e.g. 15% by wt, 20% by wt, 25% by wt, 30% by wt or 35% by wt. The term “about” is used herein to denote a variance from the listed amount of up to about 10% higher or lower from the listed amount.

In one embodiment, the orally administrable formulation comprises a polymer base comprising 50-80% by wt of polymer base comprising one or more polysaccharide-based polymers with or without a non-polysaccharide polymer in an amount of 10% or less by wt of the polymer base such as hydroxyl propyl methyl cellulose. Polysaccharides for use include as pullulan, amylose, high amylose starch, hydroxypropylated high amylose starch, alginate, pectin, dextrin, dextran, chitin, chitosan, levan, elsinan, scleroglucan and alternant. Preferred polysaccharides includes pullulan, starch, alginate, pectin, dextran and dextrin. The base may additionally include a secondary film forming agent such as xanthan gum, tragacanth gum, guar gum, acacia gum, arabic gum, carrageenan gum and/or gelatin in an amount of about 5-10% by wt of the base.

The pharmaceutical component of the present formulation comprises the selected pharmaceutical agent(s), e.g. pain medication (NSAID and/or opioid), psychedelic compound and/or cannabinoid, and an absorption enhancer, is then combined with the polymer base. The pharmaceutical agent(s) and the absorption enhancer can be combined together and added to the polymer base, or can each be added separately to the polymer base. The present absorption enhancer(s) function to permit absorption of the combination of pharmaceutical agents to achieve the synergistic effect thereof.

In one embodiment, an enzymatic absorption enhancer is incorporated within the formulation. Examples of enzymatic absorption enhancers for use in the present formulation include, but are not limited to, lipases such as pancreatic lipase (PL), pancreatic lipase-related protein 1 or 2 (PLRP1/PLRP2), hepatic lipase, endothelial lipase, lipoprotein lipase, lysosomal lipase, gastric lipase and lingual lipase. The enzyme is included in the formulation in an amount in the range of 25-170 U per litre, or 0.01-10% by wt of the pharmaceutical component, or 0.05-5% by wt of the pharmaceutical component.

In another embodiment, a non-enzymatic absorption enhancer may be utilized. An example of a non-enzymatic absorption enhancer is a bile acid. Examples of suitable bile acids for use in the present formulation include, but are not limited to, cholic acid, or a cholic acid derivative such as deoxycholic, glycocholic, chenodeoxycholic, taurocholic, glycodeoxycholic and taurodeoxycholic acid, or salts thereof and mixtures thereof. Examples of salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as those derived from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. Other salts include sulfate, citrate, phosphate and tartrate. A preferred bile acid salt for use is sodium deoxycholate.

Alternatively, or additionally, to further enhance absorption, the present formulation may include one or more non-enzymatic absorption enhancers such as: polyoxyethylene ethers, esters or alcohols; alkali metal alkyl sulfates, e.g. comprising a C8 to C22 alkyl, preferably C12 alkyl (lauryl) and any alkali metal, e.g. sodium or potassium, such as sodium lauryl sulphate; lecithin, hyaluronic acid, pharmaceutically acceptable salts of hyaluronic acid, octylphenoxypolyethoxyethanol, glycolic acid, lactic acid, oleic acid, linoleic acid, linolenic acid, monoolein, monooleates, monolaurates, borage oil, evening of primrose oil, chamomile extract, cucumber extract, menthol, trihydroxy oxo cholanylglycine, lysine, polylysine, triolein or polidocanol alkyl ethers.

As used herein, the term “polyoxyethylene ethers” (also referred to as polyethylene glycols) includes, but is not limited to, any of several condensation polymers of ethylene glycol, for example, HOCH₂ (CH₂OCH₂), CH₂OH(OCH₂CH₂), H(OCH₂CH₂) or OH (OCH₂CH₂) with average molecular weights from 200 to 6000. Also suitable are polyoxyethylene alcohols and esters. Examples of suitable compounds include Brij™ compounds, i.e., Brij™ 30, 52, 56, 58, 72, 76, 700, 721, 92, 93, 96, 97, 98, 99, etc. Polyoxyethylene ethers are preferred, and most preferred is polyoxyethylene 9-lauryl ether.

Examples of lecithin include, Phospholipon-H™ saturated phospholipid, Phospholipon-G™ unsaturated phospholipid, phosphatidylcholine, phosphatidyl serine, sphingomyelin, phosphatidylethanolamine, cephalin, and lysolecithin.

In a further embodiment, an enzymatic absorption enhancer may be combined with a non-enzymatic absorption enhancer. For example, the enzymatic absorption enhancer may be combined with a bile acid or salt thereof, or another non-enzymatic absorption enhancer such as a polyoxyethylene ether, ester or alcohol; alkali metal alkyl sulfate; hyaluronic acid, or other absorption enhancer as detailed herein, to enhance absorption of the one or more pharmaceutical agents in the formulation.

The amount of absorption enhancer for use in the present formulation may be in the range of 1-50% by weight, such as 10-40% by weight, 5-30% by weight or 1-10% by weight of the formulation.

The present orally administrable formulation is prepared by mixing the selected polymer base, and any other base components such as secondary film forming agent(s), plasticizer(s), resin(s), softener(s), filler(s) and waxes, with heat in a sufficient amount of water (q.s), such as an amount sufficient to dissolve the hydrophilic polymer base components, to attain a clear solution. The polymer base is then combined with the pharmaceutical component including the pharmaceutical agent (in the form being used, e.g. extracted form or an unextracted form such as plant or fungal material), absorption enhancer, and other desired ingredients as will be described.

The present formulation may optionally include one or more buffers which facilitate release of pharmaceutical agent from the particles and enhances bioavailability of the pharmaceutical agent, e.g. cannabinoids, terpenes and/or derivatives thereof, in the oral cavity. Examples of suitable buffers include a carbonate, such as monocarbonate, bicarbonate or sesquicarbonate; glycerinate; phosphate; glycerophosphate; acetate; glyconate or citrate of an alkali metal, such as potassium and sodium, e.g. trisodium and tripotassium citrate, ammonium, tris buffer, amino acids, and mixtures thereof. The buffer may be microencapsulated or otherwise coated as granules with polymers and/or lipids being less soluble in saliva than the buffer. Such microencapsulation controls the dissolution rate of the buffer to permit it to effectively facilitate pharmaceutical agent release. Buffer is generally included in an amount of about 5% by wt or less of the formulation.

The formulation may comprise one or more of the following additional additives: a humectant, inorganic salts, antioxidants, protease inhibitors, emulsifiers, or colorants. Non-limiting examples of humectants include propylene glycol or glycerol. Examples of inorganic salts include sodium, potassium, calcium and zinc salts, especially sodium chloride, potassium chloride, calcium chloride, zinc chloride and sodium bicarbonate Examples of antioxidants include tocopherol, deteroxime mesylate, methyl paraben, ethyl paraben, ascorbic acid and mixtures thereof. Examples of protease inhibitors include but are not limited to bacitracin and bacitracin derivatives such as bacitracin methylene disalicylates, soybean trypsin, and aprotinin. Examples of emulsifiers include lecithins (e g. E322, E342), polyglycerol polyridnoleate (e.g. PGPR, E476), citric acid esters (e.g E472c), ammoniumphosphatide (e g. E442), polyoxyethylene emulsifier (e.g. TWEEN 80), sulphate emulsifiers (e.g. SLS, SDS, SLES) and sorbitan tristearate (e g. STS, E492). Such additional additives may comprise combined between about 1 to 5 wt/wt % of the composition. Bacitracin and its derivatives preferably comprise between 1.5 and 2 wt/wt % of the total composition, while soybean trypsin and aprotinin preferably comprise between about 1 and 2 wt/wt % of the total composition.

The formulation may include an anti-microbial agent. In one embodiment, the formulation comprises one or more essential oils that confer antimicrobial properties. Preferably, the amount of a selected essential oil for use in the formulation is sufficient to provide antimicrobial efficacy while not changing the physical characteristics of the formulation, e.g. an amount ranging from 0.01 to 15 wt % (but may exceed this range). Generally, an oil such as thymol, methyl salicylate and/or eucalyptol may be present in an amount of about 0.01 to about 4 wt % of the formulation, preferably about 0.50 to about 3.0 wt % of the formulation, and even more preferably from about 0.70 to about 2.0 wt % of the formulation. Menthol may be added in an amount ranging from about 0.01 to about 15 wt % of the formulation, preferably about 2.0 about 10 wt %, and even more preferably from about 3 to about 9 wt % of the formulation. The appropriate amount of a selected anti-microbial oil in the formulation can readily be determined by one of skill in the art.

Saliva stimulating agents may be added to the formulation according to the present invention. Examples of saliva stimulating agents include food acids such as citric, lactic, malic, succinic, ascorbic, adipic, fumaric and tartaric acids. Preferred food acids are citric, malic and ascorbic acids. The amount of saliva stimulating agents suitable for inclusion in the present formulation may range from about 0.01 to about 12 wt %, preferably about 1 wt % to about 10 wt %.

The formulation may also include one or more additional agents, each in an amount of about 1-5% by wt of the formulation. Examples include solubilization agents; charge modifying agents, pH control agents, modulatory agents of epithelial junction physiology, such as nitric oxide (NO) stimulators, chitosan, or chitosan derivatives, vasodilator agents, selective transport-enhancing agents; stabilizing delivery vehicles, carriers, supports or complex-forming species with which exendin(s) is/are effectively combined, associated, contained, encapsulated or bound to stabilize the active agent for enhanced mucosal delivery; small hydrophilic penetration enhancers; emulsifiers, mucolytic or mucus clearing agents (e.g. mucoadhesive and mucosal delivery-enhancing agents); membrane penetration-enhancing agents such as e.g., (i) an alcohol, (ii) an enamine, (iii) an NO donor compound, (iv) a long-chain amphipathic molecule, (v) a small hydrophobic penetration enhancer, (vi) sodium or a salicylic acid derivative, (vii) a glycerol ester of acetoacetic acid, (viii) a cyclodextrin or beta-cyclodextrin derivative, (ix) a medium-chain fatty acid, (x) a chelating agent, (xi) an amino acid or salt thereof, (xii) an N-acetylamino acid or salt thereof, (xiii) an inhibitor of fatty acid synthesis, (xiv) an inhibitor of cholesterol synthesis; or (xv) any combination of the membrane penetration enhancing agents of (i)-(xv).

Examples of suitable mucoadhesives or mucosal delivery-enhancing agents as enhancers include Carbopol 934+HPC, Maize+Carbopol 907, HPC (hydroxypropyl cellulose), CMC or Na-CMC (carboxymethylcellulose), HPMC (hydroxypropylmethylcellulose), HEMA (hydroxyethyl metacrylate), Carbopol 907 crosslinked with sucrose, polyacrylic acids (PAA), chitosans, lectins, polymethacrylate derivatives, hyaluronic acid, P(AA-co-PEG) monomethylether monomethacrylate, PAA-PVP (poly acrylic acid-poly vinyl pyrrolidone), PVP-PEG (polyethylene glycol), methylcellulose, pullulan. N-trimethyl chitosans, PDMAEMA (poly(dimethyl-aminoethyl methacrylate)), HEC (hydroxyethyl cellulose), Carbomer 940, Carbomer 971, polyethylene oxide, dextrin, poly(methyl vinyl ether/maleic anhydride), Polycarbophil (acrylic acid crosslinked with divinyl glycol), PVP (poly vinyl pyrrolidone), agar, tragacanth, sodium alginate, karaya gum, MEC (methylethyl cellulose). HPC (hydroxy propyl cellulose), lectins, AB block copolymer of oligo (methyl methacrylate) and PAA, polymers with thiol groups, spheromers, thiomers, alginic acid sodium salt, Carbopol 974P (Carbomer), EC (ethylcellulose), dextran, guar gum, pectins, starch, gelatin, casein, acrylic acid polymers, polymers of acrylic acid esters, acrylic acid copolymers, vinyl polymers, vinyl copolymers, polymers of vinyl alcohols, alkoxy polymers, polyethylene oxide polymers, and polyethers in one embodiment, exendin is combined with one, two, three, four or more of the mucosal delivery-enhancing agents recited above.

The present formulation may also include an antifoaming agent in an amount effective to provide the desired anti-foaming effect. Commonly used antifoaming agents are insoluble oils, polydimethylsiloxanes and other silicones, certain alcohols, stearates and glycols. The additive is used to prevent formation of foam or is added to break a foam already formed.

The formulation may comprise one or more flavoring agents selected from the group consisting of essential oils, essences, extracts, powders, acids, coconut, coffee, chocolate, vanilla, grape fruit, orange, lime, menthol, liquorice, caramel aroma, honey aroma, peanut, walnut, cashew, hazelnut, almonds, pineapple, strawberry, raspberry, apple, pear, peach, apricot, blackberry, cherry, pineapple, plum essence, clove oil, bay oil, anise, thyme, cedar leaf oil, nutmeg, cinnamon, menthol, peppermint, wintergreen, spearmint, eucalyptus, mint, or any combination thereof. The formulation may also optionally comprise bright leaf, burley-leaf, oriental-leaf tobacco, Dark air cured Burley, Flue cured Virginia, and dark fired Kentucky.

The formulation may also include sweeteners, such as bulk sweeteners, sugar sweeteners, sugar substitute sweeteners, artificial sweeteners, high-intensity sweeteners, or any combination thereof. Suitable bulk sweeteners include both sugar and non-sugar sweetening components. Bulk sweeteners may constitute from about 5 to about 95% by weight of the present oral formulation. In some cases, the amount of sweetener may be about 20 to about 80% by weight, such as 30 to 70% or 30 to 60% by weight of the formulation. However, an amount may be selected to achieve the desired sweetening affect. Useful sugar sweeteners are saccharide-containing components commonly known in the art including, but not limited to, sucrose, dextrose, maltose, dextrins, trehalose, D-tagatose, dried invert sugar, fructose, levulose, galactose, corn syrup solids, and the like, alone or in combination sugar substitutes include, but are not limited to, sorbitol, mannitol, xylitol, hydrogenated starch hydrolysates, maltitol, isomalt, erythritol, lactitol and the like.

The present formulation advantageously provides an effective method of administering a pharmaceutical agent, such as an analgesic, cannabinoid, terpene and/or psychedelic drug, or mixtures thereof, which addresses one or more of the barriers associated with oral formulations of such drugs including low permeability, susceptibility to degradation, rapid clearance, and chemical instability in the gastrointestinal (GI) tract. Due to the inclusion of an absorption enhancer, including enzymatic, non-enzymatic or combinations thereof, solubility of the pharmaceutical agent is enhanced, which improves absorption of the pharmaceutical agent by the oral mucosa when it is released from the formulation on dissolution within the oral cavity, and thereby augments bioavailability of the pharmaceutical agent. The formulation also helps to mask any undesirable taste of the selected pharmaceutical agent.

The present formulation also provides a means to administer multiple pharmaceutical agents at the same time so as to enhance or otherwise improve the efficacy of one or more of the pharmaceutical agents. This is particularly beneficial for use with pain medications to enhance efficacy by combining medications that treat pain using different modes of action. This may also result in the use of reduced dosages which is relevant with pain medications such as opioids to prevent opioid dependence.

Embodiments of the invention are described by reference to the following specific example which is not to be construed as limiting.

### Example 1

This Example describes the preparation of a dissolvable orally administrable formulation in accordance with an embodiment of the invention. The following ingredients were mixed in boiling water maintained at 90-100° C. with high speed stirring (e.g. 2000 rpm) to form a polymer base gel:

| | | |
|---|---|---|
| INGREDIENT | AMOUNT (gm) | % wt |

| | | | |
|---|---|---|---|
| Na Alginate | 15 | gm | 2.0 |
| Hydroxy propyl methyl cellulose | 5 | gm | 0.7 |
| Pullulan | 25 | gm | 3.4 |
| Xanthum gum | 3 | gm | 0.4 |
| Carragenan gum | 3 | gm | 0.4 |
| Glycerin | 30 | gm | 4.0 |

| | | |
|---|---|---|
| Water | q.s | 85.2 |

When a clear solution is obtained, the following further ingredients were slowly added with high speed stirring at high temperature (70-90° C.):

| INGREDIENT | AMOUNT (gm) | |
|---|---|---|
| Tween 80 | 2 | gm |
| SDS | 1 | gm |
| Bile Acid (Na deoxycholate) | 1 | gm |
| Antifoam | 2 | gm |
| Coconut oil | 5 | gm |
| Sucralose | 7 | gm |
| Stevia | 7 | gm |
| Peppermint | 0.5 | gm |
| Mint | 0.5 | gm |
| Psilocybin | 0.04 | gm |
| Blue No. 1 (Brilliant Blue) | 0.1 | mg |
| Lingual lipase | 0.05 | gm |

Stirring was continued at high speed for 30 mins at high temp. The mixture was examined for lumps, and the stirrer was directed towards the lumps to break the lumps and generate a homogeneous solution or slurry. The mixture was cooled to room temperature with high speed stirring. The cooled solution was spread uniformly on a belt equipped with heaters and doctors and were bled to cast a film of uniform thickness. The film was determined to be soluble in the oral cavity.

### Example 2

Another orally administrable formulation according to the invention is provided below. The polymer ingredients were first admixed with water as described above to form a polymer base gel:

| | | |
|---|---|---|
| Pectin/Gelatin mixture | 5 | g |
| Pullulan | 50 | g |
| Water | 650 | g |
| Glycerin | 9 | g |

Once a clear solution was obtained, the following ingredients, including the pharmaceutical agent, were added and stirred with heating to yield a homogeneous solution or slurry.

| | | |
|---|---|---|
| Color (red) solution | 0.8 | g |
| Oil Corn | 5 | g |
| Peppermint Oil | 1 | g |
| Peppermint Flavor | 1.5 | g |
| Lecithin | 0.5 | g |
| SLS | 1 | g |
| Brij 30 | 0.5 | g |
| Lipase | 0.5 | g |
| Psilocybin | 0.04 | g |
| Sweetener (Stevia or Xylitol) | 3 | g |
| Bile acid | 0.1 | g |

The solution was then cooled to form a film that is soluble in the mouth. The film may be prepared for storage or into dosage forms for administration.

### Example 3

This Example describes the preparation of a dissolvable orally administrable formulation in accordance with an embodiment of the invention. The following polymer base ingredients are mixed (% by wt of the polymer base) in boiling water maintained at 90-100° C. with high speed stirring (e.g. 2000 rpm) to form a polymer base gel:

| | | |
|---|---|---|
| Pullulan and optionally additional | 50-80% | by wt |
| polysaccharide-based polymers | | |
| Non-polysaccharide-based hydrophilic polymer | 1-10% | by wt |
| Glycerin | 10-40% | by wt |

Once a clear solution is obtained, the pharmaceutical component comprising the following main ingredients (% by wt of the pharmaceutical component) is added and stirred with heating to yield a homogeneous solution or slurry, along with flavouring, sweetening and colouring agents as desired:

| | | |
|---|---|---|
| Lipase absorption enhancer | 0.1-0.5% | by wt |
| Bile acid | 0.1-5% | by wt |
| Other non-enzymatic absorption enhancers | 5-25% | by wt |
| Psilocybin | 0.1-1% | by wt |

A multi-modal formulation based on the above is prepared using the above formulation comprising psilocybin combined with a second pharmaceutical agent, for example, pain medication, at a reduced dosage, e.g. ¼-½ of a regular dosage, to minimize undesirable side effects including, but not limited to, dependence or addiction. This is particularly useful to reduce dosages of opioid analgesics such as hydromorphone, oxycodone, codeine, morphine and fentanyl. While the dosing for each opioid is different, exemplary dosing for hydromorphone is 2-4 mg dosages 4-5 times daily, which is reduced in the present multi-modal formulation to 0.5-2 mg dosages 2-3 times daily or less, to result in a synergistic effect in which a significantly lower dose of opioid analgesic is effective for pain relief.

### Example 4

A multi-modal formulation is prepared based on Example 3. The formulation includes as the non-polysaccharide polymer is hydroxyl propyl methyl cellulose, and the non-enzymatic absorption enhancers include an alkali metal alkyl sulfate and a polyoxyethylene ether. The pharmaceutical component includes 25 mg of diclofenac, 20 mg psilocybin, and 20 mg of morphine, or 4 mg of hydromorphone. The formulation is found to exhibit sufficient oral solubility and absorption.

### Example 5

The multi-modal formulation of Example 4 is modified to incorporate a cannabinoid. The pharmaceutical component includes 25 mg diclofenac, 10 mg psilocybin, 20 mg CBD, and 2 mg hydromorphone. The formulation is found to exhibit sufficient oral solubility and absorption.

In view of the description provided herein, aspects of the invention include, but are not limited to, the following:
i) An orally administrable formulation which dissolves in the oral cavity comprising a hydrophilic polymer base and a pharmaceutical component comprising one or more pharmaceutical agents and an absorption enhancer:
ii) The formulation of i), wherein the hydrophilic polymer base comprises one or more hydrophilic elastomeric polymers selected from the group consisting of polyethylene glycol, polyacrylamides, polyacrylic acid, acrylic acid, polyacrylic acid copolymer, polyvinyl alcohol, hydroxypropylmethyl cellulose, hydroxyethyl cellulose, hydroxypropyl cellulose, polyvinyl pyrrolidone, carboxymethyl cellulose, glycolide, polylactide, methylmethacrylate copolymer, carboxyvinyl polymer, polysaccharides such as pullulan, amylose, high amylose starch, hydroxypropylated high amylose starch, alginate, carrageenan, pectin, dextrin, dextran, chitin, chitosan, levan, elsinan, scleroglucan and alternant, and combinations thereof.
iii) The formulation of ii), wherein the hydrophilic polymer base additionally comprises a secondary film forming agent selected from xanthan gum, tragacanth gum, guar gum, acacia gum, arabic gum, carrageenan gum, collagen, gelatin, zein, gluten, soy protein isolate, whey protein isolate, casein and mixtures thereof
iv) The formulation of i), wherein the absorption enhancer is an enzyme selected from the group consisting of pancreatic lipase (PL), pancreatic lipase-related protein 1 or 2 (PLRP1/PLRP2), hepatic lipase, endothelial lipase, lipoprotein lipase, lysosomal lipase, gastric lipase and lingual lipase.
v) The formulation of i), wherein the absorption enhancer comprises an enzymatic absorption enhancer combined with a non-enzymatic absorption enhancer.
vi) The formulation of v), wherein the non-enzymatic absorption enhancer is a bile acid or salt thereof.
vii) The formulation of vi), wherein the bile acid or salt thereof is selected from the group consisting of cholic acid, deoxycholic acid, glycocholic acid, chenodeoxycholic acid, taurocholic acid, glycodeoxycholic acid and taurodeoxycholic acid, or salts or mixtures thereof.
viii) The formulation of v), wherein the non-enzymatic absorption enhancer is selected from the group of polyoxyethylene ethers, esters or alcohols; alkali metal alkyl sulfates, lecithin, hyaluronic acid, pharmaceutically acceptable salts of hyaluronic acid, octylphenoxypolyethoxyethanol, glycolic acid, lactic acid, oleic acid, linoleic acid, linolenic acid, monoolein, monooleates, monolaurates, borage oil, evening of primrose oil, chamomile extract, cucumber extract, menthol, trihydroxy oxo cholanylglycine, lysine, polylysine, triolein, polidocanol alkyl ethers and mixtures thereof.
ix) The formulation of i), comprising the pharmaceutical agent(s) in an amount in the range of 0.1-40% by wt.
x) The formulation of i), wherein the pharmaceutical agent is a psychedelic compound selected from the group consisting of a tryptamine, a phenethylamine, ketamine, ibotenic acid, muscimol, salvinorin A, lysergamide, mescaline, ibogaine and mixtures thereof.
xi) The formulation of i), wherein the pharmaceutical agent is selected from the group consisting of DMT (N,N-dimethyltryptamine), 5-hydroxy-N,N-dimethyltryptamine, 5-methoxy-N,N-dimethyltryptamine, LSD (lysergic acid diethylamide), mescaline (3,4,5-trimethoxyphenethylamine), PCP (phenylcyclohexyl piperidine), 4-phosphoryloxy-N,N-dimethyltryptamine (psilocybin), [3-[2-(methylamino)ethyl]-1H-indol-4-yl] dihydrogen phosphate (baeocystin), 4-hydroxy-N,N-dimethyltryptamine (psilocin), amphetamine, 3,4-methylenedioxy amphetamine (MDA), methylenedioxyethylamphetamine (MDEA), MDMA (3,4-methylenedioxy-methamphetamine), and a mixture thereof.
xii) The formulation of i), wherein the pharmaceutical agent is a psychedelic compound or compounds in the form of an extract from a fungus or mixture of fungi of the genera Copelandia, Gymnopilus, Inocybe, Mycena, Panaeolus, Pholiotina, Pluteus or Psilocybe.
xiii) The formulation of i), wherein the pharmaceutical agent is in the form of an extract from cacti.
xiv) The formulation of i), wherein the pharmaceutical agent is a cannabinoid selected from the group consisting of cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), naphthoylindoles; phenylacetylindoles; benzoylindoles; cyclohexylphenoles; HU-210, 3-dimethylnepty 11 carboxylic acid homologine 8, delta-9 tetrahydrocannabinol (THC or dronabinol), delta-8 tetrahydrocannabinol (D8-THC), tetrahydrocannabinol acid (THCA), tetrahydrocannabivarin (THCV), tetrahydrocannabivarin acid (THCVA), nabilone, rimonabant (SR141716), JWH-018, JWH-073, CP-55940, dimethylheptylpyran, HU-210, HU-331, SR144528, WIN 55, 212-2, JWH-133, levonantradol, AM-2201 and combinations thereof.
xv) The formulation of xiv), wherein the cannabinoid is CBD, THC or a mixture thereof.
xvi) The formulation of i), wherein the pharmaceutical agent is psilocybin, and the enzymatic absorption enhancer comprises lingual lipase and sodium deoxycholate.
xvii) The formulation of i), wherein the polymer base comprises a film-forming agent selected from pullulan, hydroxyl propyl methyl cellulose, alginate and combinations thereof, and a plasticizer comprising glycerin.
xviii) The formulation of i), wherein the pharmaceutical agent is psilocybin, the absorption enhancer comprises lingual lipase and sodium deoxycholate, and wherein the base comprises a film-forming agent selected from pullulan, hydroxyl propyl methyl cellulose, alginate and combinations thereof, and a plasticizer comprising glycerin.
xix) The formulation of i), wherein the pharmaceutical component comprises an analgesic compound in combination with one or more additional pharmaceutical agents selected from a cannabinoid, a terpene and a psychedelic drug.
xx) The formulation of i), comprising a hydrophilic polymer in an amount in the range of 10-60% by wt of the formulation, a pharmaceutical component in an amount of 0.1-40% by wt of the formulation and enzymatic absorption enhancer in an amount in the range of 25-170 U per litre of the formulation.

1. An orally administrable formulation which dissolves in the oral cavity comprising a hydrophilic polymer base and a pharmaceutical component comprising one or more pharmaceutical agents and an absorption enhancer.

2. The formulation of claim 1, wherein the hydrophilic polymer base comprises one or more hydrophilic elastomeric polymers selected from the group consisting of polyethylene glycol, polyacrylamides, polyacrylic acid, acrylic acid, polyacrylic acid copolymer, polyvinyl alcohol, hydroxypropylmethyl cellulose, hydroxyethyl cellulose, hydroxypropyl cellulose, polyvinyl pyrrolidone, carboxymethyl cellulose, glycolide, polylactide, methylmethacrylate copolymer, carboxyvinyl polymer, polysaccharides such as pullulan, amylose, high amylose starch, hydroxypropylated high amylose starch, alginate, carrageenan, pectin, dextrin, dextran, chitin, chitosan, levan, elsinan, scleroglucan and alternant, and combinations thereof.

3. The formulation of claim 1, wherein the hydrophilic polymer base comprises a polysaccharide selected from the group consisting of pullulan, amylose, high amylose starch, hydroxypropylated high amylose starch, alginate, carrageenan, pectin, dextrin, dextran, chitin, chitosan, levan, elsinan, scleroglucan and alternant, and combinations thereof.

4. The formulation of claim 1, wherein the hydrophilic polymer base additionally comprises a secondary film forming agent selected from xanthan gum, tragacanth gum, guar gum, acacia gum, arabic gum, carrageenan gum, collagen, gelatin, zein, gluten, soy protein isolate, whey protein isolate, casein and mixtures thereof.

5. The formulation of claim 1, wherein the absorption enhancer is an enzyme selected from the group consisting of pancreatic lipase (PL), pancreatic lipase-related protein 1 or 2 (PLRP1/PLRP2), hepatic lipase, endothelial lipase, lipoprotein lipase, lysosomal lipase, gastric lipase and lingual lipase.

6. The formulation of claim 1, wherein the absorption enhancer comprises an enzymatic absorption enhancer combined with a non-enzymatic absorption enhancer.

7. The formulation of claim 6, wherein the non-enzymatic absorption enhancer is a bile acid or salt thereof.

8. The formulation of claim 7, wherein the bile acid or salt thereof is selected from the group consisting of cholic acid, deoxycholic acid, glycocholic acid, chenodeoxycholic acid, taurocholic acid, glycodeoxycholic acid and taurodeoxycholic acid, or salts or mixtures thereof.

9. The formulation of claim 6, wherein the non-enzymatic absorption enhancer is selected from the group of bile acid, polyoxyethylene ethers, esters or alcohols; alkali metal alkyl sulfates, lecithin, hyaluronic acid, pharmaceutically acceptable salts of hyaluronic acid, octylphenoxypolyethoxyethanol, glycolic acid, lactic acid, oleic acid, linoleic acid, linolenic acid, monoolein, monooleates, monolaurates, borage oil, evening of primrose oil, chamomile extract, cucumber extract, menthol, trihydroxy oxo cholanylglycine, lysine, polylysine, triolein, polidocanol alkyl ethers and mixtures thereof.

10. The formulation of claim 1, wherein the pharmaceutical agent is selected from the group consisting of an anti-inflammatory agent, an opioid analgesic, a psychedelic drug, a cannabinoid and a terpene.

11. The formulation of claim 1, wherein the pharmaceutical component comprises two or more pharmaceutical agents.

12. The formulation of claim 11, wherein the pharmaceutical component comprises a multi-modal combination of pharmaceutical agents selected from the group consisting of an anti-inflammatory agent, an opioid analgesic, a psychedelic drug, a cannabinoid and a terpene.

13. The formulation of claim 11, wherein the multi-modal combination results in a reduction in the dosage of at least one of the pharmaceutical agents in comparison to the dosage of the at least one pharmaceutical agent when used alone.

14. The formulation of claim 12, comprising psilocybin.

15. The formulation of claim 12, comprising a cannabinoid selected from CBD, THC and a mixture thereof.

16. The formulation of claim 12, comprising an opioid analgesic.

17. The formulation of claim 1, wherein the pharmaceutical agent is psilocybin, and the enzymatic absorption enhancer comprises lingual lipase and sodium deoxycholate.

18. The formulation of claim 1, wherein the polymer base comprises a film-forming agent selected from pullulan, hydroxyl propyl methyl cellulose, alginate and combinations thereof, and a plasticizer comprising glycerin.

19. The formulation of claim 1, wherein the pharmaceutical agent is psilocybin, the absorption enhancer comprises lingual lipase and sodium deoxycholate, and wherein the base comprises a film-forming agent selected from pullulan, hydroxyl propyl methyl cellulose, alginate and combinations thereof, and a plasticizer comprising glycerin.

20. The formulation of claim 1, wherein the polymer base comprises pullulan and optionally additional polysaccharide-based polymers in an amount of about 50-80% by wt of the polymer base and glycerin in an amount of about 10-40% by wt of the polymer base; and the pharmaceutical component comprises a lipase absorption enhancer in an amount of about 0.1-0.5% by wt, bile acid in an amount of about 0.1-5% by wt and psilocybin in an amount of about 0.1-1% by wt, each of the pharmaceutical component.
